(19) United States
(12) Patent Application Publication
(10) Pub. No.: US 2024/0052011 A1
(43) Pub. Date: Feb. 15, 2024

(54) DOSING REGIMEN FOR GP100-SPECIFIC TCR - ANTI-CD3 SCFV FUSION PROTEIN

(71) Applicant: Immunocore Limited, Abingdon (GB)

(72) Inventor: Christina COUGHLIN, Philadelphia, PA (US)

(21) Appl. No.: 18/489,854

(22) Filed: Oct. 19, 2023

Related U.S. Application Data
(63) Continuation of application No. 16/305,838, filed on Nov. 29, 2018, now Pat. No. 11,827,688.

(30) Foreign Application Priority Data
Jun. 2, 2016 (GB) 1609683.6
Jul. 13, 2016 (GB) 1612193.1
Jul. 14, 2016 (GB) 1612260.8

(51) Int. Cl. C07K 14/725; A61P 35/00; A61K 31/4439; A61K 38/17; A61K 39/395; C07K 16/28

(52) U.S. Cl. CPC C07K 14/7051; A61P 35/00; A61K 31/4439; A61K 38/1774; A61K 39/3955; C07K 16/2809; C07K 16/2818; C07K 16/2827; A61K 2039/505

(57) ABSTRACT

The present invention relates to the treatment of cancer, particularly gp100 positive cancers. In particular, it relates to a dosage regimen for a T cell redirecting bispecific therapeutic comprising a targeting moiety that binds the YLEPGPVTA (SEQ ID NO:1)-HLA-A2 complex fused to a CD3 binding T cell redirecting moiety.

### CROSS-REFERENCE TO RELATED APPLICATIONS

This application is a continuation of U.S. application Ser. No. 16/305,838, filed Nov. 29, 2018, which is the National Stage of International Application No. PCT/GB2017/051596, filed Jun. 2, 2017, which claims the benefit of and priority to Great Britain Patent Application Serial Nos. 1609683.6, filed on Jun. 2, 2016, 1612193.1, filed Jul. 13, 2016, and 1612260.8, filed on Jul. 14, 2016, the contents of which are incorporated by reference in their entirety.

### SEQUENCE LISTING

The instant application contains a Sequence Listing which has been filed electronically in XML format and is hereby incorporated by reference in its entirety. Said XML copy, created on Oct. 13, 2023, is named 56652US_Sequence_Listing.txt and is 11,392 bytes in size.

The present invention relates to the treatment of cancer, particularly gp100 positive cancers. In particular, it relates to a dosage regimen for a T cell redirecting bispecific therapeutic comprising a targeting moiety that binds the YLEPGPVTA (SEQ ID NO:1)-HLA-A2 complex fused to a CD3 binding T cell redirecting moiety.

The human glycoprotein 100 (gp100) is one of a panel of melanoma-associated antigens to which the body can mount a natural immune response. The protein is a 661 amino acid melanosomal membrane-associated glycoprotein which is expressed in normal melanocytes and widely overexpressed on the majority of melanoma cancer cells. For example, one study (Trefzer et al., (2006) Melanoma Res. 16(2): 137-45) found that 82% of 192 melanoma metastases from 28 melanoma patients expressed gp100. Several studies reported higher expression levels of gp100 in melanoma tissues (Hofbauer et al., (2004) J Immunother. 27(1): 73-78, Barrow et al., (2006) Clin Cancer Res. 12:764-71). Whilst the majority of gp100 positive cancers are melanomas, the literature also contains numerous reports of other forms of cancer such as clear cell sarcoma and various brain cancer subtypes that express gp100 as detected for instance by immunohistochemistry with an antibody called HMB-45, a widely used gp100 specific diagnostic antibody (Huang et al., (2015) Int J Clin Exp Pathol. 8(2):2171-5, Ozuguz et al., (2014) Indian Dermatol Online J 5(4): 488-90, Taddei et al., Appl Immunohistochem Mol Morphol (2001) 9(1):35-41). The exact function of the protein is unknown but it appears to be involved in melanosome maturation (Hoashi et al., 2005; Kawakami and Rosenberg, 1997).

The gp100 antigen has been, and continues to be, the target of a number of immunotherapy-based melanoma clinical trials.

Early gp100 targeted immunotherapy involved vaccination strategies with peptides or viruses expressing the gp100 protein in its entirety, or portions thereof (reviewed in (Lens (2008) Expert Opin Biol Ther. 8(3): 315-323.)). These trials have had limited success which is thought to be a result of the generation of an insufficient immune response rather than a reflection on the validity of the target. Therefore, the principle of using gp100 as a melanoma target remains a valid approach for melanoma specific immunotherapy.

The development of a novel bispecific therapeutic comprising a T cell receptor-based, gp100 specific, targeting moiety fused to a CD3 binding T cell redirecting moiety provides a new treatment option (Liddy et al., (2012). Nat Med. 8: 980-987). The mechanism of action of such a therapeutic is significantly different to other immunotherapy predecessors and results in the rapid and potent redirection of non-gp100 specific T cells to kill gp100 positive cells in vitro; thus there is a sound rationale for expecting both improved clinical efficacy results in patients and on-target, off-tumour toxicities from activity against gp100 positive normal tissues such as skin melanocytes.

The peptide YLEPGPVTA (SEQ ID No: 1) corresponds to amino acid residue numbers 280-288 of gp100. The YLEPGPVTA (SEQ ID No: 1) peptide is presented by Class I HLA molecules on gp100+/HLA-A*02+ cancer cells (Salgaller et al., (1996) Cancer Res 56: 4749-4757). WO2011/001152 describes TCRs which bind the YLEPGPVTA (SEQ ID NO:1) peptide presented as a peptide-HLA-A*02 complex. The TCRs are mutated relative to a native gp100 TCR alpha and/or beta variable domains to have improved binding affinities for, and/or binding half-lives for the complex, and can be associated (covalently or otherwise) with a therapeutic agent. One such therapeutic agent is an anti-CD3 antibody, or a functional fragment or variant of said anti-CD3 antibody such as a single chain variable fragment (scFv). The anti-CD3 antibody or fragment may be covalently linked to the C- or N- terminus of the alpha or beta chain of the TCR. Such TCRs have been used to treat melanoma in patients (clinical trial identifier NCT01211262).

IMCgp100 is a T cell redirecting bispecific therapeutic agent comprising a soluble affinity enhanced TCR that binds to the YLEPGPVTA (SEQ ID NO:1) peptide-HLA-A*02 complex, fused to an anti-CD3 scFv. IMCgp100 has been shown to function in vitro by binding to gp100 positive target cells and inducing killing via the recruitment of both tumour-specific and non-tumour-specific T cells. Once bound to the peptide-HLA-A*02 complex on the surface of a gp100 positive target cell, IMCgp100 crosslinks the targeted cell to the T cell through the interaction of the anti-CD3 scFv portion of the molecule. Crosslinking of the two cells via the bi-specific therapeutic results in the formation of an immune synapse, activation of the T cell and redirection of a T cell response against the gp100 positive target cell. IMCgp100 can activate both CD4⁺ and CD8+ T cells; however, its primary function is to induce CD8+ T cell responses. CD8+ T cells, also known as cytotoxic T lymphocytes (CTL), play an essential role in the natural immune system's repertoire for fighting cancer. CTLs are cytolytic and function to induce lysis of the tumour cell through the apoptotic pathway.

As mentioned above, gp100 is expressed in normal healthy cells including melanocytes, which raises the possibility of on-target, off-tumour reactivity in a clinical setting. Expression levels of gp100 in melanocytes are known to be lower than in tumours. During preclinical testing of IMCgp100, it was confirmed that IMCgp100 was able to redirect T cell activity against normal melanocytes, but at a lower potency than tumour cells, potency in this context being directly proportional to the number of epitopes displayed on the surface of the target cells (Liddy et al., (2012). Nat Med. 8: 980-987). While these data indicate a potential therapeutic window between on-tumour and off-tumour reactivity, on-target, off-tumour reactivity cannot be excluded with IMCgp100 treatment. Any reactivity to melanocytes in the skin during clinical testing would be expected to manifest as skin-related toxicity such as rash, vitiligo and the effects of localised cytokine and chemokine release, which may be managed with hydrocortisone treatment. No other safety concerns were identified during in vitro preclinical testing of IMCgp100, including no evidence for off-target cross reactivity. IMCgp100 was approved for clinical testing based on this data. As will be appreciated by those skilled in the art, suitable therapeutic dose levels cannot be predicted a priori and depend on both the mechanism of action of the drug and the expression profile of the target antigen. It therefore follows that, whilst the on-target, off-tumour toxicities of numerous gp100 specific vaccines and autologous T cell therapies that have been evaluated in the clinic are known, it would still be impossible to predict an appropriate therapeutic dose for a T cell redirecting therapeutic a priori. This is because, whilst vaccines and autologous T cell therapies result in only a small proportion of T cells in the patient being able to recognise the gp100 positive cells, a T cell redirecting therapeutic can in theory provide every CD3 positive T cell in the patient the capacity to recognise and react to gp100 positive cells.

The safety and tolerability of IMCgp100 in patients with advanced melanoma has been investigated in a Phase I trial (clinical trial identifier NCT01211262). Interim results of this study have been presented (Middleton et al., (2015) Cancer Res 2015;75 (15 Suppl):Abstract nr CT106). The maximum tolerated dose for weekly administration was determined to be 600ng/kg, which was transitioned to a recommended phase II dose (RP2D) of 50 μg per dose, irrespective of the patient's weight. During the dose escalation portion of the study, severe (grade ¾) hypotension was observed as the main dose limiting toxicity. A cohort of patients subsequently received weekly dosing at RP2D and further episodes of severe hypotension were reported. Other common adverse events included rash, pruritus, pyrexia and oedema. Adverse events typically manifested shortly after the first or second dose of drug. Additional weekly doses of IMCgp100 beyond dose 2 resulted in fewer adverse reactions and a general amelioration in the severity of these events. To increase tolerability during early doses the first weekly dose of IMCgp100 was reduced by 20% to 40 μg as is common practice, followed by dosing at RP2D in subsequent weeks. However, cases of severe hypotension continued to occur. Expression of gp100 in melanocytes within the skin provides a large sink of antigen positive cells in the largest organ in the body. Potent targeting of gp100 positive cells by IMCgp100 leads to redirected T cell activity against gp100 positive skin melanocytes. This manifests as skin-related toxicity in patients. Analysis of patient serum and biopsy samples demonstrate localised cytokine and chemokine release within both skin and tumours resulting in a high volume of T cells being trafficked from the peripheral circulation into tissues within an extremely short space of time; the substantial fluid shifts associate with this rapid T cell migration result in hypotension.

In a first aspect, the present invention provides a T cell redirecting bispecific therapeutic which comprises (i) a targeting moiety that binds the YLEPGPVTA (SEQ ID NO:1)-HLA-A2 complex fused to (ii) a CD3 binding T cell redirection moiety, for use in a method of treating gp100 positive cancer in a patient comprising administering the bispecific therapeutic to said patient, wherein each dose is administered every 5-10 days, at least the first and second doses are below 40 μg and the second dose is higher than the first dose.

The inventors have surprisingly found an intra-patient dose escalation regimen that provides improved tolerability for treating gp100 positive tumours with a bi-specific therapeutic employing a T cell redirection mediated mechanism-of-action. The present invention provides an escalating dosage regimen in which undesirable side effects, such as severe hypotension, are significantly reduced. The dosage regimen is useful to reduce the severity of on-target, off-tumour toxicity in patients thereby reducing the need for patients to remain hospitalised and/or be admitted to intensive care during the first few weeks of treatment. An additional and surprising benefit is that, through ameliorating the effects on on-target, off-tumour T cell activity by use of the dosing regimen, patients can subsequently safely tolerate higher overall therapeutic dose levels than the existing R2P2 of 50 μg, which is expected to result in improved clinical efficacy. Without wishing to be bound by theory, the inventors hypothesise that the dose limiting toxicity of hypotension is driven by the Cₘₐₓ exposure levels of the bi-specific therapeutic, the presence of a large amount of gp100 positive melanocytes within the skin compartment and the extent of tumour gp100 expression within the patient with respect to both the patient's disease burden and the expression level of gp100 within the patients tumours. The inventors believe that the dosing regimen may allow sufficient destruction of normal skin melanocytes expressing high levels of gp100 to subsequently allow safer administration of higher therapeutic doses which result in improved targeting of remaining gp100 positive tumour cells and thereby improve clinical efficacy.

The inventors observed that patients with tumours with larger diameters or higher overall disease burden experienced objective tumour responses at higher overall exposures to IMCgp100 than patients with smaller tumours or disease burden. Based on this data, the inventors hypothesise that an increased exposure to drug in the weeks following mitigation of the previously dose limiting toxicities through use of the dosing regimen of the invention may lead to an enhanced tumour response.

In the present invention, the bispecific therapeutic may be administered as follows:
(a) at least one first dose in the range of from 10-30 μg;
(b) at least one second dose in the range of from 20-40 μg, wherein the or each second dose is higher than the first dose; and then
(c) at least one dose of at least 50 μg.

In the present invention, the respective doses may be expressed as a specified weight of therapeutic irrespective of the patient's weight or whether the same amount of therapeutic would be administered if calculated through one of the other methods routinely used to calculate an appropriate dosage for a patient such as weight of therapeutic per Kg of body weight, body surface area or lean muscle mass etc. It is preferred if the specified weight of therapeutic is administered at weekly intervals, e.g. on days 1, 8, 15, 22, etc of the treatment regimen but the dosing interval could be longer or shorter. The dosing interval may depend on the antigen binding characteristics of the T cell redirecting bispecific therapeutic and its clearance half-life. Thus, each dose may be administered every 6-9 or 6-8 days. Preferably each dose is administered every 7 days. The respective doses may be separated by different intervals. Alternatively, they may be separated by the same interval.

The first dose may be in the range of from 10-30 μg. It may be in the range of from 13-27 μg, 15-25 μg or 18-22 μg. The dose may be 15, 16, 17, 18, 19, 20, 21, 22, 23, 24 or 25 μg. One preferred first dose is 20 μg, which may be administered on a weekly basis. One first dose may be administered. Alternatively, more than one first dose, preferably 2-5 first doses and more preferably two first doses, may be administered. It is preferred that, if two or more first doses are administered, they are the same. However, they may be different.

The second dose may be in the range of from 20-40 μg and may be higher than the first dose. The second dose may be 1, 2, 3, 4, 5, 6, 7, 8, 9 or 10 μg higher than the first dose. It may be in the range of from 23-37 μg, 25-35 μg or 28-32 μg. The subsequent dose may be 25, 26, 27, 28, 29, 30, 31, 32, 33, 34 or 35 μg. One preferred second dose is 30 μg, which may be administered on a weekly basis. One second dose may be administered. Alternatively, more than one second dose, preferably 2-5 second doses and more preferably two second doses, may be administered. It is preferred that, if two or more second doses are administered, they are the same. However, they may be different.

The dose after the second dose may be at least 50 μg. It may be in the range of from 50-300 μg, 50-250 μg, 50-200 μg, 50-150 μg, 50-100 μg, 50-90 μg, 50-80 μg, 50-70 μg or 50-60 μg. The dose may be at least 50, 55, 60, 65, 70, 75, 80, 85, 90, 95, 100, 110, 120, 130, 140, 150, 160, 170, 180, 190, 200, 210, 220, 230, 240 or 250 μg. One preferred dose after the second dose is 50 μg. Another is 60 μg and a yet further is 70 μg. The same dose may be used subsequently. Alternatively, the dose may be escalated. For example, the dose may be 5, 10, 15, 20, 30, 40 or 50 μg higher.

The first dose may be 20 μg, the second dose may be 30 μg and/or the dose after the second dose may be 50 μg or greater.

The T cell redirecting bispecific therapeutic useful in the present invention comprises (i) a targeting moiety that binds the YLEPGPVTA (SEQ ID NO:1)-HLA-A2 complex fused to (ii) a CD3 binding T cell redirection moiety.

The targeting moiety may be a T cell receptor (TCR). TCRs are described using the International Immunogenetics (IMGT) TCR nomenclature, and links to the IMGT public database of TCR sequences. The unique sequences defined by the IMGT nomenclature are widely known and accessible to those working in the TCR field. For example, they can be found in the “T cell Receptor Factsbook”, (2001) LeFranc and LeFranc, Academic Press, ISBN 0-12-441352-8; Lefranc, (2011), Cold Spring Harb Protoc2011(6): 595-603; Lefranc, (2001), Curr Protoc Immunol Appendix 1: Appendix 10; Lefranc, (2003), Leukemia 17(1): 260-266, and on the IMGT website (www.IMGT.org)

The TCRs useful in the invention may be in any format known to those in the art. For example, the TCRs may be αβ heterodimers, or they may be in single chain format (such as those described in WO9918129). Single chain TCRs include αβ TCR polypeptides of the type: Vα-L-Vβ, Vβ-L-Vα, Vα-Cα-L-Vβ, Vα-L-Vβ-Cβ or Vα- Cα-L-Vβ-Cβ, optionally in the reverse orientation, wherein Vα and Vβ are TCR a and p variable regions respectively, Cα and Cβ are TCR a and p constant regions respectively, and L is a linker sequence. The TCR is preferably in a soluble form (i.e. having no transmembrane or cytoplasmic domains). For stability, such soluble TCRs preferably have an introduced disulfide bond between residues of the respective constant domains, as described, for example, in WO 03/020763. Preferred TCRs of this type include those which have a TRAC1 constant domain sequence and a TRBC1 or TRBC2 constant domain sequence except that Thr 48 of TRAC1 and Ser 57 of TRBC1 or TRBC2 are replaced by cysteine residues, the said cysteines forming a disulfide bond between the TRAC1 constant domain sequence and the TRBC1 or TRBC2 constant domain sequence of the TCR. It may though contain full length alpha and beta chains.

The alpha and/or beta chain constant domain may be truncated relative to the native/naturally occurring TRAC/TRBC sequences. In addition the TRAC/TRBC may contain modifications. For example, the alpha chain extracellular sequence may include a modification relative to the native/naturally occurring TRAC whereby amino acid T48 of TRAC, with reference to IMGT numbering, is replaced with C48. Likewise, the beta chain extracellular sequence may include a modification relative to the native/naturally occurring TRBC1 or TRBC2 whereby S57 of TRBC1 or TRBC2, with reference to IMGT numbering, is replaced with C57. These cysteine substitutions relative to the native alpha and beta chain extracellular sequences enable the formation of a non-native interchain disulphide bond which stabilises the refolded soluble TCR, i.e. the TCR formed by refolding extracellular alpha and beta chains (WO 03/020763). This non-native disulphide bond facilitates the display of correctly folded TCRs on phage (Li et al., Nat Biotechnol 2005 Mar;23(3):349-54). In addition the use of the stable disulphide linked soluble TCR enables more convenient assessment of binding affinity and binding half-life. Alternative positions for the formation of a non-native disulphide bond are described in WO 03/020763.

TCRs useful in the invention may be engineered to include mutations. Methods for producing mutated high affinity TCR variants such as phage display and site directed mutagenesis and are known to those in the art (for example see WO 04/044004 and Li et al., Nat Biotechnol 2005 Mar;23(3):349-54).

TCRs useful in the present invention may have a binding affinity for, and/or a binding half-life for, the YLEPGPVTA (SEQ ID NO:1)-HLA-A2 complex at least double that of a TCR having the extracellular alpha chain sequence SEQ ID No: 2 and the extracellular beta chain sequence SEQ ID No: 3. TCRs useful in the invention may have a K_{D} for the complex of ≤8 μM, ≤55 μM, ≤1 μM, ≤50.1 μM, ≤50.01 μM, ≤50.001 μM, or 50.0001 μM and/or have a binding half-life (T½) for the complex of ≥1.5 s, ≥3 s, ≥10 s, ≥20 s, ≥40 s, ≥60 s, ≥600 s, or ≥6000 s. A preferred K_{D} and/or binding half-life (T½) are approximately 10-100 μM and approximately 6-48 hours respectively. A particularly preferred K_{D} and/or binding half-life (T½) are approximately 24 μM and approximately 24 hours.

Binding affinity (inversely proportional to the equilibrium constant K_{D}) and binding half-life (expressed as T½) can be determined by any appropriate method. It will be appreciated that doubling the affinity of a TCR results in halving the K_{D}. T½ is calculated as In2 divided by the off-rate (k_{off}). So doubling of T½ results in a halving in k_{off}. K_{D} and k_{off} values for TCRs are usually measured for soluble forms of the TCR, i.e. those forms which are truncated to remove hydrophobic transmembrane domain residues. Therefore it is to be understood that a given TCR meets the requirement that it has a binding affinity for, and/or a binding half-life for, the YLEPGPVTA (SEQ ID NO:1)-HLA-A2 complex if a soluble form of that TCR meets that requirement. Preferably the binding affinity or binding half-life of a given TCR is measured several times, for example 3 or more times, using the same assay protocol, and an average of the results is taken. In a preferred embodiment these measurements are made using the Surface Plasmon Resonance (BIAcore) method of Example 3 of WO2011/001152. The reference gp100 TCR—having the extracellular alpha chain sequence SEQ ID No: 2 and the extracellular beta chain sequence SEQ ID No: 3—has a K_{D} of approximately 19 μM as measured by that method, and a k_{off} of approximately 1 s⁻¹ (i.e T½ of approximately 0.7 s).

The TCRs useful in the invention may be associated with an anti-CD3 antibody, or a functional fragment or variant of said anti-CD3 antibody, and may be as described in WO2011/001152. Antibody fragments and variants/analogues which are suitable for use in the compositions and methods described herein include but are not limited to minibodies, Fab fragments, F(ab′)₂ fragments, dsFv and scFv fragments, Nanobodies™ (these constructs, marketed by Ablynx (Belgium), comprise synthetic single immunoglobulin variable heavy domain derived from a camelid (e.g. camel or llama) antibody) and Domain Antibodies (Domantis (Belgium), comprising an affinity matured single immunoglobulin variable heavy domain or immunoglobulin variable light domain).

TCR-anti CD3 fusions useful in the invention include a TCR alpha chain amino acid sequence selected from the group consisting of:
(i) the TCR alpha chain sequence of SEQ ID No: 2, wherein amino acids 1 to 109 are replaced by the sequence of SEQ ID No: 4, wherein amino acid at position 1 is S;
(ii) the TCR alpha chain sequence of SEQ ID No: 2, wherein amino acids 1 to 109 are replaced by the sequence of SEQ ID No: 4, wherein amino acid at position 1 is A;
(iii) the TCR alpha chain sequence of SEQ ID No: 2, wherein amino acids 1 to 109 are replaced by the sequence of SEQ ID No: 4, wherein amino acid at position 1 is G;
(iv) the TCR alpha chain sequence of SEQ ID No: 2, wherein amino acids 1 to 109 are replaced by the sequence of SEQ ID No: 4, wherein amino acid at position 1 is S, and the C-terminus of the alpha chain is truncated by 8 amino acids from F196 to S203 inclusive, based on the numbering of SEQ ID No: 2;
(v) the TCR alpha chain sequence of SEQ ID No: 2, wherein amino acids 1 to 109 are replaced by the sequence of SEQ ID No: 4, wherein amino acid at position 1 is A, and the C-terminus of the alpha chain is truncated by 8 amino acids from F196 to S203 inclusive, based on the numbering of SEQ ID No: 2;
(vi) the TCR alpha chain sequence of SEQ ID No: 2, wherein amino acids 1 to 109 are replaced by the sequence of SEQ ID No: 4, wherein amino acid at position 1 is G, and the C-terminus of the alpha chain is truncated by 8 amino acids from F196 to S203 inclusive, based on the numbering of SEQ ID No: 2; and

a TCR beta chain-anti-CD3 amino acid sequence selected from the group consisting of:
(vii) the TCR beta chain-anti-CD3 sequence of SEQ ID No: 5, wherein amino acids at positions 1 and 2 are D and I respectively;
(viii) the TCR beta chain-anti-CD3 sequence of SEQ ID No: 5, wherein amino acids at positions 1 and 2 are A and I respectively;
(ix) the TCR beta chain-anti-CD3 sequence of SEQ ID No: 5, wherein amino acids at positions 1 and 2 are A and Q respectively;
(x) the TCR beta chain-anti-CD3 sequence of SEQ ID No: 5, wherein amino acids at positions 1 and 2 are D and I respectively amino acids at positions 108-131 are replaced by RTSGPGDGGKGGPGKGPGGEGTKGTGPGG (SEQ ID No: 6), and amino acids at positions 254-258 are replaced by GGEGGGSEGGGS (SEQ ID No: 7);
(xi) the TCR beta chain-anti-CD3 sequence of SEQ ID No: 5, wherein amino acids at positions 1 and 2 are D and I respectively and amino acid at position 257 is a S and amino acid at position 258 is a G;
(xii) the TCR beta chain-anti-CD3 sequence of SEQ ID No: 5, wherein amino acids at positions 1 and 2 are D and I respectively and amino acid at position 256 is a S and amino acid at position 258 is a G;
(xiii) the TCR beta chain-anti-CD3 sequence of SEQ ID No: 5, wherein amino acids at positions 1 and 2 are D and I respectively and amino acid at position 255 is a S and amino acid at position 258 is a G;
(xiv) the TCR beta chain-anti-CD3 sequence of SEQ ID No: 5, wherein amino acids at positions 1 and 2 are A and Q, and wherein amino acid at position 257 is a S and amino acid at position 258 is a G;
(xv) the TCR beta chain-anti-CD3 sequence of SEQ ID No: 5, wherein amino acids at positions 1 and 2 are A and Q, and wherein amino acid at position 256 is a S and amino acid at position 258 is a G;
(xvi) the TCR beta chain-anti-CD3 sequence of SEQ ID No: 5, wherein amino acids at positions 1 and 2 are A and Q, and wherein amino acid at position 255 is a S and amino acid at position 258 is a G;
(xvii) the TCR beta chain-anti-CD3 sequence of SEQ ID No: 5, wherein amino acid at positions 1 and 2 are A and I respectively, and wherein amino acid at position 257 is a S and amino acid at position 258 is a G;
(xviii) the TCR beta chain-anti-CD3 sequence of SEQ ID No: 5, wherein amino acid at positions 1 and 2 are A and I respectively, and wherein amino acid at position 256 is a S and amino acid at position 258 is a G;
(xix) the TCR beta chain-anti-CD3 sequence of SEQ ID No: 5, wherein amino acid at positions 1 and 2 are A and I respectively, and wherein amino acid at position 255 is a S and amino acid at position 258 is a G;

### Examples of Such TCR-Anti CD3 Fusions are a TCR in which

the alpha chain amino acid sequence is (i) and the beta chain-anti-CD3 amino acid sequence is (vii);
the alpha chain amino acid sequence is (i) and the beta chain-anti-CD3 amino acid sequence is (x);
the alpha chain amino acid sequence is (vi) and the beta chain-anti-CD3 amino acid sequence is (ix);
the alpha chain amino acid sequence is (v) and the beta chain-anti-CD3 amino acid sequence is (viii);
the alpha chain amino acid sequence is (vi) and the beta chain-anti-CD3 amino acid sequence is (vii);
the alpha chain amino acid sequence is (i) and the beta chain-anti-CD3 amino acid sequence is (xi);
the alpha chain amino acid sequence is (i) and the beta chain-anti-CD3 amino acid sequence is (xii);
the alpha chain amino acid sequence is (i) and the beta chain-anti-CD3 amino acid sequence is (xiii);
the alpha chain amino acid sequence is (vi) and the beta chain-anti-CD3 amino acid sequence is (xiv);
the alpha chain amino acid sequence is (vi) and the beta chain-anti-CD3 amino acid sequence is (xv);
the alpha chain amino acid sequence is (vi) and the beta chain-anti-CD3 amino acid sequence is (xvi);
the alpha chain amino acid sequence is (v) and the beta chain-anti-CD3 amino acid sequence is (xvii);
the alpha chain amino acid sequence is (v) and the beta chain-anti-CD3 amino acid sequence is (xviii);
the alpha chain amino acid sequence is (v) and the beta chain-anti-CD3 amino acid sequence is (xix);
the alpha chain amino acid sequence is (vi) and the beta chain-anti-CD3 amino acid sequence is (xi);
the alpha chain amino acid sequence is (vi) and the beta chain-anti-CD3 amino acid sequence is (xii); and the alpha chain amino acid sequence is (vi) and the beta chain-anti-CD3 amino acid sequence is (xiii).

A preferred TCR-anti CD3 fusion has the alpha and beta chains of SEQ ID NOs: 4 and 5 respectively. A preferred TCR-anti CD3 fusion has the alpha chain amino acid sequence of (v) and the beta chain-anti-CD3 amino acid sequence of (viii).

Alternatively, the targeting moiety may be an antibody. The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term “antibody” includes antibody fragments, derivatives, functional equivalents and homologues of antibodies, humanised antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. Cloning and expression of chimeric antibodies are described in EP-A-0120694 and EP-A-0125023. A humanised antibody may be a modified antibody having the variable regions of a non-human, e.g. murine, antibody and the constant region of a human antibody. Methods for making humanised antibodies are described in, for example, U.S. Pat. No. 5,225,539. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to herein as “mAb”.

It is possible to take an antibody, for example a monoclonal antibody, and use recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin (see, for instance, EP-A-184187, GB 2188638A or EP-A-239400). A hybridoma (or other cell that produces antibodies) may be subject to genetic mutation or other changes, which may or may not alter the binding specificity of antibodies produced.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature. 1989 Oct. 12; 341(6242):544-6) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science. 1988 Oct. 21; 242(4877):423-6; Huston et al., Proc Natl Acad Sci USA. 1988 Aug;85(16):5879-83); (viii) bispecific single chain Fv dimers (PCT/US92/09965) and (ix) “diabodies”, multivalent or multispecific fragments constructed by gene fusion (WO94/13804; P. Hollinger et al., Proc Natl Acad Sci USA. 1993 Jul. 15; 90(14):6444-8). Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). Where bispecific antibodies are to be used, these may be conventional bispecific antibodies, which can be manufactured in a variety of ways (Hollinger & Winter, Curr Opin Biotechnol. 1993 August; 4(4):446-9), e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in Traunecker et al., EMBO J. 1991 December; 10(12):3655-9). Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

The binding moiety may be an TCR-like molecule that has been designed to specifically bind a peptide-MHC complex. Of particular preference are TCR-mimic antibodies, such as, for example those described in WO2007143104 and Sergeeva et al., Blood. 2011 Apr. 21; 117(16):4262-72 and/or Dahan and Reiter. Expert Rev Mol Med. 2012 Feb. 24; 14:e6.

Also encompassed within the present invention are binding moieties based on engineered protein scaffolds. Protein scaffolds are derived from stable, soluble, natural protein structures which have been modified to provide a binding site for a target molecule of interest. Examples of engineered protein scaffolds include, but are not limited to, affibodies, which are based on the Z-domain of staphylococcal protein A that provides a binding interface on two of its a-helices (Nygren, FEBS J. 2008 June;275(11):2668-76); anticalins, derived from lipocalins, that incorporate binding sites for small ligands at the open end of a beta-barrel fold (Skerra, FEBS J. 2008 Jun;275(11):2677-83), nanobodies, and DARPins. Engineered protein scaffolds are typically targeted to bind the same antigenic proteins as antibodies, and are potential therapeutic agents. They may act as inhibitors or antagonists, or as delivery vehicles to target molecules, such as toxins, to a specific tissue in vivo (Gebauer and Skerra, Curr Opin Chem Biol. 2009 June;13(3):245-55). Short peptides may also be used to bind a target protein. Phylomers are natural structured peptides derived from bacterial genomes. Such peptides represent a diverse array of protein structural folds and can be used to inhibit/disrupt protein-protein interactions in vivo (Watt, Nat Biotechnol. 2006 February;24(2):177-83)].

As described above in relation to TCRs, the CD3 binding T cell redirection moiety may be an anti-CD3 antibody, or a functional fragment or variant of said anti-CD3 antibody, and may be as described in WO2011/001152.

In one embodiment of the present invention, the method comprises administering to a patient a dose of no more than 20 μg of a T cell redirecting bi-specific therapeutic in week 1, a dose of no more than 30 μg in week 2, and a dose of at least 50 μg in week 3 and subsequent weeks, wherein the TCR moiety of the bi-specific therapeutic is associated with a T cell redirecting anti-CD3 antibody or fragment thereof and has a binding affinity for, and/or a binding half-life for, the YLEPGPVTA (SEQ ID NO:1)-HLA-A2 complex at least double that of a TCR having the extracellular alpha chain sequence SEQ ID No: 2 and the extracellular beta chain sequence SEQ ID No: 3.

It is preferred if the T cell redirecting bi-specific therapeutic is administered by intravenous infusion. Alternative routes of administration include other parenteral routes, such as subcutaneous or intramuscular infusion, enteral (including oral or rectal), inhalation or intranasal routes.

The bispecific therapeutic for use in the present invention may be administered as a monotherapy. Alternatively it may be administered in combination with one or more anti-cancer therapies, preferably immuno-modulatory therapies. The potential for additional immune activation with combination therapies increases the risk of on-target off-tumour toxicities. Such therapies include:
chemotherapy agents, such as dacarbazine and temozolamide,
immunotherapeutic agents, such as interleukin-2 (IL-2) and interferon (IFN)
checkpoint inhibitors such as agents that target PD-1 or PD-L1, e.g. pembrolizumab, nivolumab, atezolizumab, avelumab and durvalumab, and agents that target CTLA-4 such as ipilimumab and tremelimumab,
BRAF inhibitors, such as vemurafenib and dabrafenib,
MEK inhibitors, such as trametinib,
TGF-β inhibitors such as galunisertib,
MET kinase inhibitors such as merestinib.

Preferred combination therapies use durvalumab, tremelimumab, galunisertib and merestinib in combination with a T cell redirecting bi-specific therapeutic as described above. Where merestinib—see WO2010011538 or similar amidophenoxyindazole as described therein—is used in combination with the bi-specific therapeutic, the dose of merestinib may be in the range of from 40 to 120 mg once daily, preferably in the range of from 80 to 120 mg once daily.

The bispecific therapeutic may be administered on its own for the first and subsequent doses, with the additional therapeutic agents being added thereafter or vice-versa. In embodiments of the present invention where a T cell redirecting bispecific therapeutic and another cancer therapy are administered in combination, the T cell redirecting bispecific therapeutic may be administered alone in weeks 1 and 2 and the other therapy added in week 3 and subsequent weeks.

Combination therapies may lead to increased immune activation including cytokine and chemokine secretion and lymphocyte trafficking, and thus increased risk of side effects such as severe hypotension. Accordingly, the dose of a T cell redirecting bispecific therapeutic may be initially given as single agent prior to combination dosing. In a preferred embodiment a dose of no more than 20 μg is administered in week 1, a dose of no more than 30 μg is administered in week 2, and a dose of at least 50 μg is administered in week 3 and subsequent weeks. Dosing of one or more additional immuno-modulatory therapies may be administered from week 3.

The present invention relates to the treatment of gp100 positive cancers. One such cancer is melanoma. Malignant melanoma continues to represent a major public health problem globally. It is estimated that almost 76,000 new diagnoses of melanoma will be made in 2016 in the United States and almost 10,000 people are expected to die of melanoma (American Cancer Society Statistics, 2016). In addition, the rate of diagnosis of melanoma continues to rise at a significant rate; according to 2015 Surveillance, Epidemiology, and End Results data, the average incidence rate rose 1.4% each year for the last decade, and melanoma is now the fifth most common cancer diagnosis in the United States. While the prognosis for patients with early stage disease is good following surgical resection, the median survival rate drops rapidly with disease progression, falling to less than one year for patients with distal metastatic disease (Garbe et al., Oncologist 2011; 16:5-24). The rising incidence of melanoma combined with poor outcomes with standard chemotherapy and early signals from immune-based therapy have led to intense investigation of novel approaches and combinations to enhance the anti-tumour immune response.

The melanoma to be treated in accordance with the present invention may be cutaneous melanoma or uveal melanoma (also known as ocular melanoma). Alternatively, it may be any one of Lentigo maligna melanoma, superficial spreading melanoma, ascral lentiginous melanoma, mucosal melanoma, nodular melanoma, polypoid melanoma, desmoplastic melanoma, amelanotic melanoma, soft-tissue melanoma, small cell melanoma with small nevus-like cells and spitzoid melanoma.

The melanoma to be treated is generally late-stage disease (advanced disease), typically characterised by metastatic lesions. For example, Stage Ill and/or Stage IV, (Balch et al., J Clin Oncol 2009; 27:6199-206).

Uveal melanoma (UM) is a rare type of melanoma that is a biologically distinct from cutaneous melanoma. UM is an extremely malignant neoplasm that affects the vascular layers of the eye (iris, ciliary body, and choroid). Despite its rare incidence rate (representing approximately 3% of melanoma cases, approximately 4,000 cases globally per year), (Papastefanou et al., (2011) J Skin Cancer;2011:573974). The suppressive environment and lack of activity of checkpoint inhibition in UM suggests that mobilization of activated T cells with a tumour-specific focus may have anti-tumour activity in this disease setting.

As mentioned above other non-melanoma cancers have been reported in the literature as being gp100 positive, examples being clear cell sarcoma and neurologic cancers such as some gliomas. The present invention can be used to treat such cancers.

In a further aspect, the present invention provides a method of treating gp100 positive cancer in a patient comprising administering a T cell redirecting bispecific therapeutic to said patient, wherein each dose is administered every 5-10 days, at least the first and second doses are below 40 μg and the second dose is higher than the first dose.

In a further aspect, the present invention provides a T cell redirecting bispecific therapeutic which comprises (i) a targeting moiety that binds the YLEPGPVTA (SEQ ID NO:1)-HLA-A2 complex fused to (ii) a CD3 binding T cell redirection moiety, for use in a method of treating gp100 positive cancer in a patient comprising the following sequential steps:
(a) administering to the patient a weekly dose in the range of from 10-30 μg of the bispecific therapeutic;
(b)(i) if the patient experiences grade 1 or lower treatment-related hypotension, increasing the dose administered to the patient to a weekly dose in the range of from 20-40 μg of the bispecific therapeutic, or
(b)(ii) if the patient experiences grade 2 or higher treatment-related hypotension, continuing administering to the patient a weekly dose in the range of from 10-30 μg of the bispecific therapeutic until the patient experiences grade 1 or lower treatment-related hypotension and then increasing the dose administered to the patient to a weekly dose in the range of from 20-40 μg of the bispecific therapeutic; and
(c)(i) if the patient experiences grade 1 or lower treatment-related hypotension, increasing the dose administered to the patient to a weekly dose of at least 50 μg of the bispecific therapeutic or
(c)(ii) if the patient experiences grade 2 or higher treatment-related hypotension, continuing administering to the patient a weekly dose in the range of from 20-40 μg of the bispecific therapeutic until the patient experiences grade 1 or lower treatment-related hypotension and then increasing the dose administered to the patient to a weekly dose of at least 50 μg of the bispecific therapeutic.

In this aspect, the method may further comprise, between steps (b) and (c), the step of:
(d)(i) if the patient experiences grade 1 or lower treatment-related hypotension, increasing the dose administered to the patient to a weekly dose in the range of from 30-45 μg of the bispecific therapeutic, or
(d)(ii) if the patient experiences grade 2 or higher treatment-related hypotension, continuing administering to the patient a weekly dose in the range of from 20-40 μg of the bispecific therapeutic until the patient experiences grade 1 or lower treatment-related hypotension and then increasing the dose administered to the patient to a weekly dose in the range of from 30-45 μg of the bispecific therapeutic.

The grade of hypotension may be assessed by a physician.

Preferred features of each aspect of the invention are as for each of the other aspects mutatis mutandis. It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the T cell redirecting bispecific therapeutic for use and method for treating gp100 positive cancer are specifically embraced by the present invention and are disclosed herein just as if each and every combination was individually and explicitly disclosed. The published documents mentioned herein are incorporated to the fullest extent permitted by law. Citation or identification of any document in this application is not an admission that such document is available as prior art to the present invention.

### REFERENCE IS MADE HEREIN TO THE ACCOMPANYING DRAWINGS IN WHICH

FIG. 1 shows the amino acid sequence of the alpha chain extracellular domain of the reference gp100 TCR (SEQ ID No; 2);

FIG. 2 shows the amino acid sequence of the beta chain extracellular domain of the reference gp100 TCR (SEQ ID No; 3);

FIG. 3 shows the amino acid sequence of a gp100-specific TCR a chain (SEQ ID No: 4);

FIG. 4 shows the amino acid sequence of an anti-CD3 scFv antibody fragment (bold type) fused via a linker, namely GGGGS (underlined), at the N-terminus of a gp100-specific TCR @chain (SEQ ID No 5);

FIG. 5 shows the occurrence of toxicities including severe and/or serious hypotension relative to the number of doses of IMCgp100;

FIG. 6 shows lymphocyte trafficking from the periphery following the first dose of IMCgp100;

FIG. 7 shows T cell proliferation with IMCgp100 in the presence or absence anti-CTLA-4;

FIG. 8 shows T cell proliferation with IMCgp100 in the presence or absence of anti- PD-L1;

FIG. 9 shows secretion of cytokines IFNγ, TNFα and IL-2 with IMCgp100 in the presence or absence of anti-CTLA-4; and

FIG. 10 shows secretion of cytokine IFNγ with IMCgp100 in the presence or absence of anti-CTLA-4.

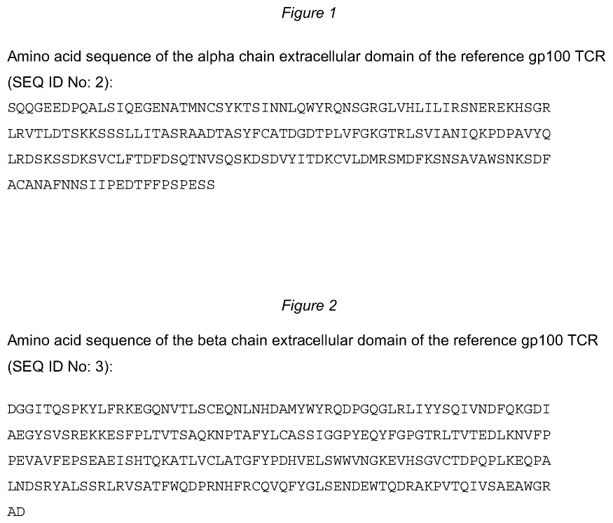

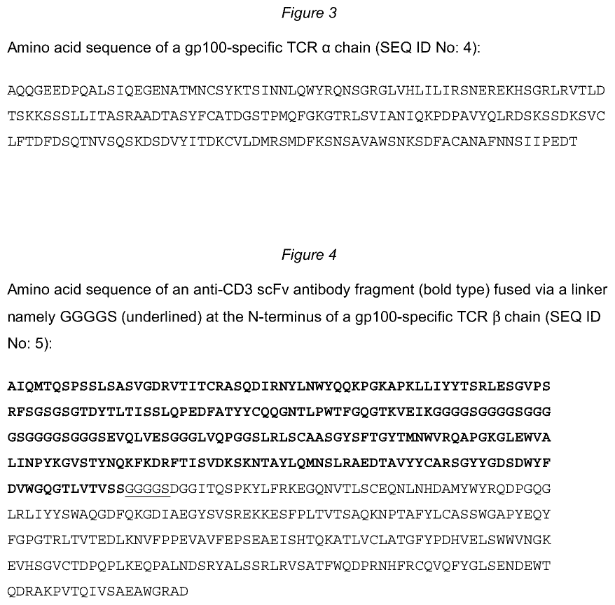

### EXAMPLES

### Example 1—Identification of a Dosage Regimen for IMCgp100 which Ameliorates Drug-Related Severe Hypotension and Allows for an Increased Upper Dose

IMCgp100 is a T cell redirecting bispecific agent comprising a soluble affinity enhanced TCR that binds to the YLEPGPVTA (SEQ ID NO:1) peptide-HLA-A*02 complex, fused to an anti-CD3 scFv. [the alpha and beta chains of IMCgp100 are SEQ ID NOs 4 and 5 respectively] IMCgp100 was investigated in an first-in-human (FIH), open-label, dose finding study to assess the safety and tolerability of IMCgp100 in patients with advanced malignant melanoma (clinical trials identifier: NCT01211262). The study was designed to identify the maximum tolerated dose (MTD) or recommended Phase II dose (RP2D) of IMCgp100 in 2 repeat dosing regimens: (Arm 1) weekly dosing (the RP2D-QW) and (Arm 2) daily dosing ×4 days (the RP2D-QD).

Patients with stage IV or un-resectable stage Ill malignant melanoma were enrolled on the study. All patients were HLA-A*02 positive, were over 18 years of age, and were classified has having measurable disease according to RECIST 1.1 criteria, with a life expectancy in excess of three months and an Eastern Cooperative Oncology Group (ECOG) performance status of 1 or below. There were no limits on the number of prior therapies. Patients were assessed for 33308/56652/FW/17875698.1 adequate haematologic, renal, hepatic, and cardiac function. Patients with symptomatic brain metastases were specifically excluded from the study. In total, 84 patients received treatment.

IMCgp100 was administered by intravenous infusion using a controlled infusion pump. Screening procedures and tests establishing eligibility were performed no more than 14 days before dosing with IMCgp100 commenced, except for HLA typing, MRI scan, ophthalmological, audiological and echocardiological assessments, which were performed within 28 days before dosing commenced. Informed consent was obtained from all patients. Blood samples for haematological, biochemical and pharmacokinetic (PK) analyses were obtained at screening, on day 1 prior to infusion, at 4, 10 and 24 hours following commencement of infusion and on days 2, 8 and 30.

Weekly Dosing: Dose Escalation Results

A standard 3+3 Phase I dose escalation protocol was followed to define the maximum tolerated dose (MTD). Briefly, the dose of IMCgp100 was escalated in cohorts of 3(+3) patients until criteria for MTD were met, or until the target limiting dose was reached. Dose escalation proceeded in three-fold increments, moving to a modified Fibonacci design according to safety and PK profile or if a dose limiting toxicity (DLT) was reported. MTD was defined as the highest dose level at which a DLT was experienced in greater than 33% of patients enrolled at that level. DLTs were observed within an 8-day window following treatment and were assessed following the NCI Common Terminology Criteria for Adverse Events (CTCAE) version 4.0. Transient Grade 3 or Grade 4 lymphopenia, and non-life threatening cutaneous skin rash were excluded as dose limiting criteria because of the anticipated pharmacological effects of the drug.

During dose escalation, 31 patients were treated across eight dose cohorts and received from 5 ng/kg to 900 ng/kg of drug. Dose limiting toxicity (DLT) of grade 3 or 4 hypotension was observed in four patients during dose escalation at 405 ng/kg (n=1 of 6), 600 ng/kg (n=1 of 6), and 900 ng/kg (n=2 of 6). In 3 of 4 DLT cases of grade 3 or 4 hypotension, the event occurred following the first dose of IMCgp100 at approximately 12-18 hours post-dose; 1 DLT hypotension event occurred with the second dose at 405 ng/kg, in a patient receiving concomitant antihypertensive therapy. Hypotension in these 4 cases was managed with IV fluids (normal saline and colloid infusions); none of the patients required pharmacologic (inotropic) support for blood pressure and all were treated with IV corticosteroid therapy. All cases of grade 3 or 4 hypotension resolved with fluid therapy and IV corticosteroid therapy within 2 days.

TABLE 1
Summary of dose limiting toxicities (DLTs) for weekly dose escalation

| Dose Level (ng/kg) | N (patients) | DLT Observed |
|---|---|---|
| 5 | 3 | — |
| 15 | 3 | — |
| 45 | 3 | — |
| 135 | 3 | — |
| 270 | 3 | — |
| 405 | 6 | One grade 3 hypotension^{a} |
| 600 | 6 | One grade 4 hypotension^{a} |
| 900 | 4 | Two grade 3 hypotension^{a} |

DLT = dose limiting toxicity.
^{a}Grade 3 and 4 hypotension was associated with a significant and rapid decrease in peripheral lymphocyte count.

Based on this data, the MTD for weekly dosing was declared at 600 ng/kg.

Weekly Dosing: RP2D-QW Cohort Results

A review of the PK and safety data from the weekly dose escalation cohorts suggested that more severe toxicities and higher drug exposures of IMCgp100 were associated with higher absolute doses administered. Based on these data and the range of absolute doses administered at MTD of 600 ng/kg (n=5 pts, range 34-66 μg QW, median dose of 54 μg), the recommended phase 2 dose of the weekly dosing regimen (designated RP2D-QW) was initially determined to be a flat dose of 50 μg administered on a weekly basis.

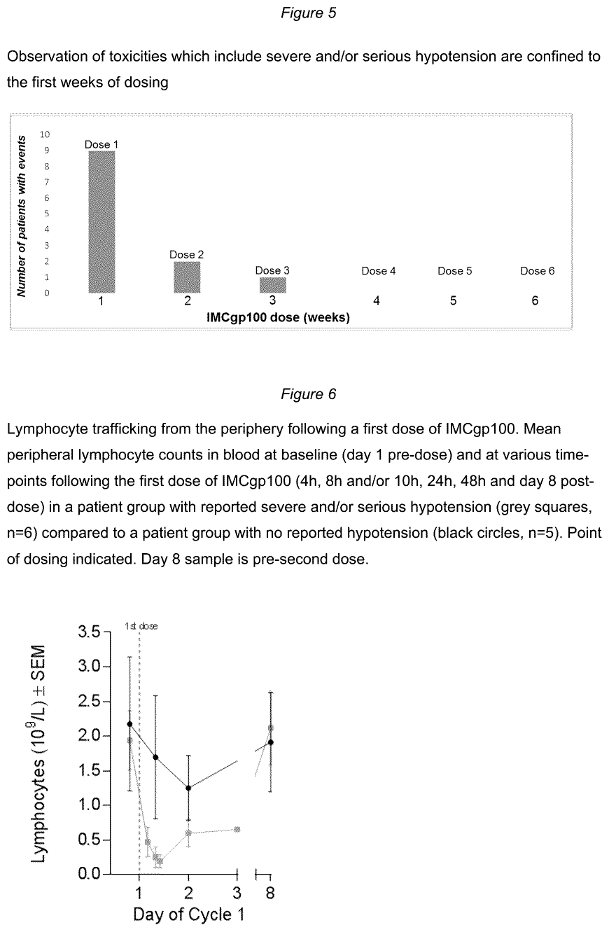

During this time, an additional three patients experienced adverse events involving grade 2 or greater hypotension. These events were observed to be confined to the first two doses (e.g. Cycle 1 Day 1 (C1D1) and Cycle 1 Day 8 (C1D8)) (FIG. 5). In an attempt to avoid severe hypotension events, the first dose of IMCgp100 was subsequently reduced to 40 μg; however, hypotension (at grade ¾) was observed in a further two patients following dosing at C1 D1 and/or C1D8; in one patient, grade 2 hypotension was also experienced following the third dose.

No cases of severe hypotension were reported following subsequent doses. It should be noted that that the inventors observed a link between the frequency and severity of hypotension and the level of gp100 expression within the patient's tumours, patients with uveal melanoma having particularly high levels of gp100 expression and therefore at higher risk of toxicity.

A further 7 patients (6 uveal and 1 cutaneous melanoma) at high risk of toxicity due to the high levels of gp100 expression within tumours were enrolled on the weekly dose expansion phase received a first dose of 20 μg (C1D1), followed by a second dose at 30 μg (C1D8), finally moving to a flat dose of 50 μg for the third dose and beyond. Surprisingly, no severe (grade ¾) hypotension was reported for these 7 patients despite their higher risk of experiencing gp100 expression mediated toxicities.

Table 2 below summarises the three dosage regimens used in the study, the number of patients treated on each regimen and the number of hypotension adverse events that were reported.

TABLE 2
Summary of weekly dosage regimens and hypotension events

| | | No. hypotension events | | |
|---|---|---|---|---|
| Weekly Regimen | No. patients | Grade 2 | Grade 3 | Grade 4 |
| Flat dose | 35 | 4 | 4 | 1 |
| (600/900 ng/kg/50 μg) | | | | |
| 40 μg (C1D1) - 50 μg | 3 | 2 | 2 | 0 |
| 20 μg (C1D1) - 30 μg | 7 | 1 | 0 | 0 |
| (C1D8) - 50 μg | | | | |

These data demonstrate the surprising benefit of a two-step intra-patient dose escalation regimen, in ameliorating drug-related severe hypotension when treating patients with gp100 positive cancers.

Hypotension results from lymphocyte trafficking and cytokine release Analysis of blood from a subset of patients dosed at weekly RP2D showed marked lymphocyte trafficking after IMCgp100 had been administered. Lymphocyte levels dropped substantially at about 12-24h post-infusion, returning to baseline levels by Day 8. This effect was more profound in cases with severe and/or serious hypotension (FIG. 6). Cytokine analyses revealed modest levels of one or more inflammatory cytokines. The greatest increases observed in the periphery were in the levels of tissue chemoattractant chemokines that parallel the transient drop in peripheral circulating lymphocytes generally resolving by 48 hours after dosing and reaching pre-dose levels by Day 8.

Further Testing of Intra-Patient Dosage Regimen

A retrospective review of the tumour response data from the patients treated in the above Phase 1 study generally noted objective responses were obtained in patients who received higher absolute doses (approximately 65-85 μg). It was therefore hypothesised that using 20 μg at C1D1 and 30 μg at C1D8 may allow for higher absolute doses, i.e. above 50 μg, at Cycle 1 Day 15 (C1D15) and beyond, and potentially improve efficacy.

To test this, IMCgp100 is being further investigated in an ongoing phase I open-label, multi-centre study in patients with advanced uveal melanoma (clinical trial identifier: NCT02570308). The first part of the study follows a standard 3+3 dose escalation design. The first cohort of patients have received IMCgp100 administered by intravenous infusion at a fixed dose of 20 μg at C1D1 and 30 μg at C1D8, followed by dosing at 60 μg from C1D15. Initial data indicate no severe hypotension-related events were reported for patients in this cohort (n=3). Further cohorts of patients will receive higher doses at C1D15 (e.g. 70 μg, 80 μg or higher).

### Example 2—Evidence for Increased Risk of Severe Hypotension for IMCgp100 Administered in Combination with Other Immune-Modulating Drugs

IMCgp100-mediated immune activation was investigated in vitro in the presence or absence of checkpoint inhibitor antibodies against PD-L1/PD-1 and CTLA-4.

Increased potency of T cell response with IMCgp100 in combination with anti-CTLA-4 In this experiment T cell proliferation was used as read-out for potency. IMCgp100 was used at a concentration of 80 μM in the presence or absence of 40 ug/ml anti-CTLA-4 (clone L3D10; Biolegend). HLA-A*02 positive monocytes pulsed with 10 nM or 100 nM gp100 peptide were used as target antigen presenting cells and plated at 10,000 cells/well. Effector CD3+ T cells were labelled with Cell Tracker Violet. T cell proliferation was measured after 5 days using the FACS based Intellicyt assay.

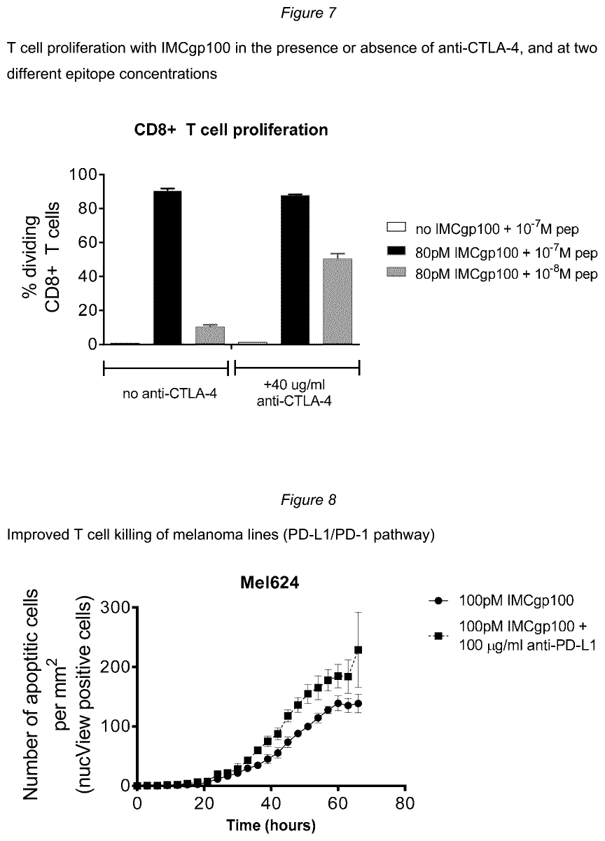

The results presented in FIG. 7 show that IMCgp100 in combination with anti CTLA-4 results in an improved T cell response (at lower peptide concentrations) relative to IMCgp100 alone, indicating a potential for higher levels of both efficacy and gp100 specific on-target, off-tumour toxicity in patients treated with this combination relative to IMCgp100 monotherapy.

Increased potency of T cell killing with IMCgp100 in combination with anti-PD-L1 In this experiment, T cell killing was used as read-out for potency. IMCgp100 was used at a concentration of 80 μM and 100 μM in the presence or absence of 10 ug/ml anti-PD-L1 (clone 29E.2A3; BioLegend). HLA-A*02 positive Me1624 cells were used as target antigen presenting cells. CD8+ T cells were used as effector cells at an effector target ratio of 5:1. T cell killing was measured using the Incucyte ZOOM assay over 3 days. Caspase- 3/7 activation in target cells was monitored as a measure of apoptosis (imaged every 2 hours).

The results presented in FIG. 8 show that IMCgp100 in combination with anti PD-L1 results in augmented T cell killing relative to IMCgp100 alone, indicating a potential for higher levels of both efficacy and gp100 specific on-target, off-tumour toxicity in patients treated with this combination relative to IMCgp100 monotherapy.

Increased Production of Pro-Inflammatory Cytokines with IMCgp100 in Combination with Anti-CTLA-4 or anti-PD-L1

In this experiment, cytokine secretion was assessed using the V-PLEX human pro-inflammatory panel 1 assay kit from Meso Scale Discovery.

For IMCgp100 in combination with anti-CTLA-4 and anti-PD-L1, supernatant samples were taken at 24h from the proliferation assay and the killing assay respectively.

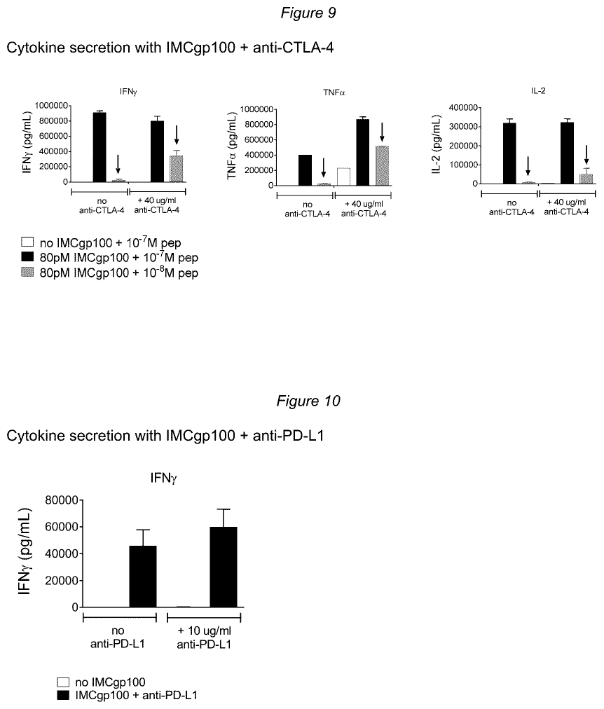

The data shown in FIG. 9 demonstrate augmented secretion of IFNγ, TNFα and IL-2 (at low peptide concentrations) in the presence of IMCgp100 and anti-CTLA-4, relative to IMCgp100 alone.

The data shown in FIG. 10 demonstrate augmented secretion of IFNγ in the presence of IMCgp100 and anti-PD-L1, relative to IMCgp100 alone.

These data demonstrate increased immune activation with IMCgp100 combination therapy relative to IMCgp100 monotherapy, and therefore a potential increased risk of severe hypotension for patients receiving the combination therapy, due to on-target off-tumour targeting of gp100 positive melanocytes.

1. A T cell redirecting bispecific therapeutic which comprises (i) a targeting moiety that binds the YLEPGPVTA-HLA-A2 complex fused to (ii) a CD3 binding T cell redirection moiety, for use in a method of treating gp100 positive cancer in a patient comprising administering the bispecific therapeutic to said patient, wherein each dose is administered every 5-10 days, at least the first and second doses are below 40 μg and the second dose is higher than the first dose.

2. The bispecific therapeutic for use of claim 1, wherein the method comprises administration of:
(a) at least one first dose in the range of from 10-30 μg;
(b) at least one second dose in the range of from 20-40 μg, wherein the or each second dose is higher than the first dose; and then
(c) at least one dose of at least 50 μg.

3. The bispecific therapeutic for use of claim 1 or claim 2, wherein each dose is administered is administered every 7 days.

4. The bispecific therapeutic for use of claim 1, 2 or 3, wherein the first dose is 20 μg, the second dose is 30 μg and/or the dose after the second dose is at least 50 μg.

5. The bispecific therapeutic for use of any preceding claim, wherein two second doses are administered.

6. The bispecific therapeutic for use of any preceding claim, wherein the targeting moiety is a T cell receptor (TCR).

7. The bispecific therapeutic for use of claim 7, wherein the TCR has a binding affinity for, and/or a binding half-life for, the YLEPGPVTA-HLA-A2 complex at least double that of a TCR having the extracellular alpha chain sequence SEQ ID No: 2 and the extracellular beta chain sequence SEQ ID No: 3.

8. The bispecific therapeutic for use of any preceding claim, wherein the CD3 binding T cell redirection moiety is anti-CD3 antibody.

9. The bispecific therapeutic for use of claim 8 or claim 9, wherein the bispecific therapeutic includes a TCR alpha chain amino acid sequence selected from the group consisting of:
(i) the TCR alpha chain sequence of SEQ ID No: 2, wherein amino acids 1 to 109 are replaced by the sequence of SEQ ID No: 4, wherein amino acid at position 1 is S;
(ii) the TCR alpha chain sequence of SEQ ID No: 2, wherein amino acids 1 to 109 are replaced by the sequence of SEQ ID No: 4, wherein amino acid at position 1 is A;
(iii) the TCR alpha chain sequence of SEQ ID No: 2, wherein amino acids 1 to 109 are replaced by the sequence of SEQ ID No: 4, wherein amino acid at position 1 is G;
(iv) the TCR alpha chain sequence of SEQ ID No: 2, wherein amino acids 1 to 109 are replaced by the sequence of SEQ ID No: 4, wherein amino acid at position 1 is S, and the C-terminus of the alpha chain is truncated by 8 amino acids from F196 to S203 inclusive, based on the numbering of SEQ ID No: 2;
(v) the TCR alpha chain sequence of SEQ ID No: 2, wherein amino acids 1 to 109 are replaced by the sequence of SEQ ID No: 4, wherein amino acid at position 1 is A, and the C-terminus of the alpha chain is truncated by 8 amino acids from F196 to S203 inclusive, based on the numbering of SEQ ID No: 2;
(vi) the TCR alpha chain sequence of SEQ ID No: 2, wherein amino acids 1 to 109 are replaced by the sequence of SEQ ID No: 4, wherein amino acid at position 1 is G, and the C-terminus of the alpha chain is truncated by 8 amino acids from F196 to S203 inclusive, based on the numbering of SEQ ID No: 2; and
a TCR beta chain-anti-CD3 amino acid sequence selected from the group consisting of:
(vii) the TCR beta chain-anti-CD3 sequence of SEQ ID No: 5, wherein amino acids at positions 1 and 2 are D and I respectively;
(viii) the TCR beta chain-anti-CD3 sequence of SEQ ID No: 5, wherein amino acids at positions 1 and 2 are A and I respectively;
(ix) the TCR beta chain-anti-CD3 sequence of SEQ ID No: 5, wherein amino acids at positions 1 and 2 are A and Q respectively;
(x) the TCR beta chain-anti-CD3 sequence of SEQ ID No: 5, wherein amino acids at positions 1 and 2 are D and I respectively amino acids at positions 108-131 are replaced by RTSGPGDGGKGGPGKGPGGEGTKGTGPGG (SEQ ID No: 6), and amino acids at positions 254-258 are replaced by GGEGGGSEGGGS (SEQ ID No: 7);
(xi) the TCR beta chain-anti-CD3 sequence of SEQ ID No: 5, wherein amino acids at positions 1 and 2 are D and I respectively and amino acid at position 257 is a S and amino acid at position 258 is a G;
(xii) the TCR beta chain-anti-CD3 sequence of SEQ ID No: 5, wherein amino acids at positions 1 and 2 are D and I respectively and amino acid at position 256 is a S and amino acid at position 258 is a G;
(xiii) the TCR beta chain-anti-CD3 sequence of SEQ ID No: 5, wherein amino acids at positions 1 and 2 are D and I respectively and amino acid at position 255 is a S and amino acid at position 258 is a G;
(xiv) the TCR beta chain-anti-CD3 sequence of SEQ ID No: 5, wherein amino acids at positions 1 and 2 are A and Q, and wherein amino acid at position 257 is a S and amino acid at position 258 is a G;
(xv) the TCR beta chain-anti-CD3 sequence of SEQ ID No: 5, wherein amino acids at positions 1 and 2 are A and Q, and wherein amino acid at position 256 is a S and amino acid at position 258 is a G;
(xvi) the TCR beta chain-anti-CD3 sequence of SEQ ID No: 5, wherein amino acids at positions 1 and 2 are A and Q, and wherein amino acid at position 255 is a S and amino acid at position 258 is a G;
(xvii) the TCR beta chain-anti-CD3 sequence of SEQ ID No: 5, wherein amino acid at positions 1 and 2 are A and I respectively, and wherein amino acid at position 257 is a S and amino acid at position 258 is a G;
(xviii) the TCR beta chain-anti-CD3 sequence of SEQ ID No: 5, wherein amino acid at positions 1 and 2 are A and I respectively, and wherein amino acid at position 256 is a S and amino acid at position 258 is a G;
(xix) the TCR beta chain-anti-CD3 sequence of SEQ ID No: 5, wherein amino acid at positions 1 and 2 are A and I respectively, and wherein amino acid at position 255 is a S and amino acid at position 258 is a G;

10. The bispecific therapeutic for use of claim 10, wherein:
the alpha chain amino acid sequence is (i) and the beta chain-anti-CD3 amino acid sequence is (vii);
the alpha chain amino acid sequence is (i) and the beta chain-anti-CD3 amino acid sequence is (x);
the alpha chain amino acid sequence is (vi) and the beta chain-anti-CD3 amino acid sequence is (ix);
the alpha chain amino acid sequence is (v) and the beta chain-anti-CD3 amino acid sequence is (viii);
the alpha chain amino acid sequence is (vi) and the beta chain-anti-CD3 amino acid sequence is (vii);
the alpha chain amino acid sequence is (i) and the beta chain-anti-CD3 amino acid sequence is (xi);
the alpha chain amino acid sequence is (i) and the beta chain-anti-CD3 amino acid sequence is (xii);
the alpha chain amino acid sequence is (i) and the beta chain-anti-CD3 amino acid sequence is (xiii);
the alpha chain amino acid sequence is (vi) and the beta chain-anti-CD3 amino acid sequence is (xiv);
the alpha chain amino acid sequence is (vi) and the beta chain-anti-CD3 amino acid sequence is (xv);
the alpha chain amino acid sequence is (vi) and the beta chain-anti-CD3 amino acid sequence is (xvi);
the alpha chain amino acid sequence is (v) and the beta chain-anti-CD3 amino acid sequence is (xvii);
the alpha chain amino acid sequence is (v) and the beta chain-anti-CD3 amino acid sequence is (xviii);
the alpha chain amino acid sequence is (v) and the beta chain-anti-CD3 amino acid sequence is (xix);
the alpha chain amino acid sequence is (vi) and the beta chain-anti-CD3 amino acid sequence is (xi);
the alpha chain amino acid sequence is (vi) and the beta chain-anti-CD3 amino acid sequence is (xii); and
the alpha chain amino acid sequence is (vi) and the beta chain-anti-CD3 amino acid sequence is (xiii).

11. The bispecific therapeutic for use of claim 11, wherein the alpha chain amino acid sequence is (v) and the beta chain-anti-CD3 amino acid sequence is (viii).

12. The bispecific therapeutic for use of any preceding claim, which is administered in combination with one or more anti-cancer therapies.

13. The bispecific therapeutic for use of claim 13, wherein the anti-cancer therapy is durvalumab, tremelimumab, galunisertib and merestinib.

14. The bispecific therapeutic for use of claim 14, wherein the anti-cancer therapy is merestinib and the dose of merestinib is in the range of from 40 to 120 mg once daily, preferably in the range of from 80 to 120 mg once daily.

15. The bispecific therapeutic for use of any preceding claim, wherein the gp100 positive cancer is melanoma.

16. A method of treating gp100 positive cancer in a patient comprising administering a T cell redirecting bispecific therapeutic to said patient, wherein each dose is administered every 5-10 days, at least the first and second doses are below 40 μg and the second dose is higher than the first dose.


SEQUENCE LISTING (7 sequences)

SEQ ID NO: 1 - protein, 9 aa, synthetic construct
  REGION 1..9: note = Amino acid residues 280-288 of gp100
YLEPGPVTA

SEQ ID NO: 2 - protein, 203 aa, synthetic construct
  REGION 1..203: note = Amino acid sequence of the alpha chain extracellular domain of the reference gp100 TCR
SQQGEEDPQALSIQEGENATMNCSYKTSINNLQWYRQNSGRGLVHLILIRSNEREKHSGRLRVTLDTSKKSSSLLITASRAADTASYFCATDGDTPLVFGKGTRLSVIANIQKPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 3 - protein, 242 aa, synthetic construct
  REGION 1..242: note = Amino acid sequence of the beta chain extracellular domain of the reference gp100 TCR
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIGGPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 4 - protein, 195 aa, synthetic construct
  REGION 1..195: note = Amino acid sequence of a gp100-specific TCR alpha chain
AQQGEEDPQALSIQEGENATMNCSYKTSINNLQWYRQNSGRGLVHLILIRSNEREKHSGRLRVTLDTSKKSSSLLITASRAADTASYFCATDGSTPMQFGKGTRLSVIANIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 5 - protein, 500 aa, synthetic construct
  REGION 1..500: note = Amino acid sequence of an anti-CD3 scFv antibody fragment fused via a linker namely GGGGS at the N-terminus of a gp100-specific TCR beta chain
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 6 - protein, 29 aa, synthetic construct
  REGION 1..29: note = Amino acid sequence to replace amino acids at positions 108-131 of SEQ ID NO: 5
RTSGPGDGGKGGPGKGPGGEGTKGTGPGG

SEQ ID NO: 7 - protein, 12 aa, synthetic construct
  REGION 1..12: note = Amino acid sequence to replace amino acids at positions 254-258 of SEQ ID No: 5
GGEGGGSEGGGS